(19) United States
(12) Patent Application Publication
(10) Pub. No.: US 2026/0229981 A1
(43) Pub. Date: Aug. 6, 2026

(54) LOW POWER MODE FOR ELECTRONIC DEVICES

(71) Applicant: Whoop, Inc., Boston, MA (US)

(72) Inventors: Luc Kearns Davidson, Acton, MA (US); Brian Anthony Martins, Somerville, MA (US); Sean Vincent Sexton, Georgetown, MA (US); Katherine Marie Kem, Somerville, MA (US)

(21) Appl. No.: 19/042,406

(22) Filed: Jan. 31, 2025

(51) Int. Cl. H02M 1/00; A61B 5/024; G01D 5/14

(52) U.S. Cl. CPC H02M 1/0032; A61B 5/02427; G01D 5/142; A61B 2560/0209; A61B 2562/242

(57) ABSTRACT

A device includes a low power shipping mode that preserves power during prolonged intervals, irreversibly transitioning into a high power operating mode upon user activation. A sensor in the device (e.g., a Hall effect sensor) may cooperate with a trigger (e.g., a magnet) in packaging to detect when the device is removed therefrom, and may initiate a transition from a low power mode to a normal power mode in response to this detection. The transition out of the low power shipping mode may include a removal or disablement of components of the low power mode, e.g., to mitigate tampering and increase reliability by preventing the device from returning to a shipping mode. Transitioning from a shipping mode to a normal operating mode can advantageously preserve battery life during shipping while ensuring quick and easy activation, thus improving the out-of-box experience and preventing unintended reentry into low power mode during use.

### TECHNICAL FIELD

The present disclosure generally relates to power management for electronic devices during shipping, and more specifically to transitioning between a low power shipping mode and a high power operating mode based on hardware triggers in packaging or the like.

### BACKGROUND

Electronic devices designed for continuous use may lack traditional on/off switches. During transit, the batteries for such devices will tend to discharge, potentially requiring charging before initial use. This can lead to a suboptimal out-of-box experience for users who might hope for immediate functionality upon unpackaging their new device. While some devices address this challenge by separately shipping batteries and devices, or by shipping devices with a physical barrier such as a removable strip of paper between battery contacts and other electronics, these approaches are difficult or impossible for environmentally sealed, continuous-use electronics such as wearable fitness monitors.

There remains a need for improved power management techniques for electronic devices that can effectively balance battery preservation after manufacture with quick and easy activation for end-users.

### SUMMARY

A device includes a low power shipping mode that preserves power during prolonged intervals, and that irreversibly transitions into a high power operating mode upon user activation. For example, a sensor in the device (e.g., a Hall effect sensor) may cooperate with a trigger (e.g., a magnet) in packaging to detect when the device is removed from the packaging, and may initiate a transition from a low power mode to a normal power mode in response to this detection. The transition out of the low power shipping mode may include a removal or disablement of components of the low power mode, e.g., to mitigate tampering and increase reliability by preventing the device from returning to a shipping mode. Transitioning from a shipping mode to a normal operating mode in this manner can advantageously preserve battery life during shipping while ensuring quick and easy activation for end-users, thus improving the out-of-box experience and preventing unintended reentry into low power mode during use.

In one aspect, a system disclosed herein may include a device having a low power mode and a normal power mode, where the normal power mode drives one or more sensors for monitoring, the low power mode provides reduced power consumption relative to the normal power mode, and the device is configured to transition from the low power mode to the normal power mode in response to a signal. The system may also include a waking module on the device, the waking module including a Hall effect sensor, the waking module configured to detect a condition for transitioning from the low power mode to the normal power mode with the Hall effect sensor, and in response to detecting the condition, to provide the signal to the device for transitioning from the low power mode to the normal power mode. The system may also include: a transition module on the device, the transition module configured to respond to the signal by permanently disabling the low power mode on the device; and packaging for delivering the device to a consumer, where the device is placed within the packaging, and where the packaging includes a disposable magnet positioned in the packaging as a sensor trigger to stimulate the Hall effect sensor to detect the condition for transitioning from the low power mode to the normal power mode when the device is removed from the packaging.

Implementations may include one or more of the following features. The device may include a wearable physiological monitor. The device may include a photoplethysmography monitor. The low power mode may use a reduced clock speed compared to the normal power mode. The low power mode may disconnect power to sensing circuitry of the device. The device may include a wireless charging interface.

In an aspect, a system disclosed herein may include: a device having a low power mode and a normal power mode, where the normal power mode drives one or more sensors for monitoring, the low power mode provides reduced power consumption relative to the normal power mode, and the device is configured to transition from the low power mode to the normal power mode in response to a signal; a waking module configured to detect a condition for transitioning from the low power mode to the normal power mode and, in response to detecting the condition, to provide the signal to the device for transitioning from the low power mode to the normal power mode; and a transition module configured to respond to the signal by transitioning to the normal power mode and permanently disabling the low power mode on the device.

Implementations may include one or more of the following features. The low power mode may include a sensing circuit containing at least one sensor different than the one or more sensors for monitoring. The low power mode may include a sensing circuit that uses at least one of the one or more sensors for monitoring. At least one of the one or more sensors may be coupled to the device in two parallel circuits including a first parallel circuit for the low power mode and a second parallel circuit for the normal power mode. At least one of the one or more sensors may include a photodetector responsive to a predetermined range of wavelengths. The one or more sensors for monitoring may include one or more sensors for physiologically monitoring a wearer of the device. The low power mode may use at least one of a reduced clock speed compared to the normal power mode. The low power mode may disconnect power to sensing circuitry of the device. The device may include a wearable photoplethysmography monitor. The device may include a battery sealed in a waterproof enclosure and a wireless charging interface for the battery. The system may further include packaging for providing the device to a user, where the device includes a Hall effect sensor, and where the packaging includes a disposable magnet positioned in the packaging as a sensor trigger to satisfy the condition for transitioning by stimulating the Hall effect sensor when the device is removed from the packaging. Permanently disabling the low power mode may include deleting code stored in a memory of the device that configures a processor of the device to execute in the low power mode. Permanently disabling the low power mode may include irreversibly and mechanically decoupling circuitry for the low power mode from the device.

In an aspect, a method disclosed herein may include: providing a device, the device including a Hall effect sensor, a processor, and a memory, the processor configured by computer executable code stored in the memory to operate the device in a low power mode and a normal power mode, and the processor configured to transition from the low power mode to the normal power mode in response to a signal from the Hall effect sensor; and placing the device in packaging, where the packaging includes a magnet positioned as a sensor trigger to generate the signal with the Hall effect sensor when the device is removed from the packaging, where the device is placed in the packaging in the low power mode, and where the device is configured, responsive to entering the normal power mode, to disable the low power mode by deleting executable code for the low power mode from the memory of the device.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system for conditionally transitioning between power modes for an electronic device using packaging thereof.

FIG. 7 is a cross-sectional view of an electronic device in its packaging.

FIG. 8 is a flow chart of a method for managing power modes of an electronic device.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary.

The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

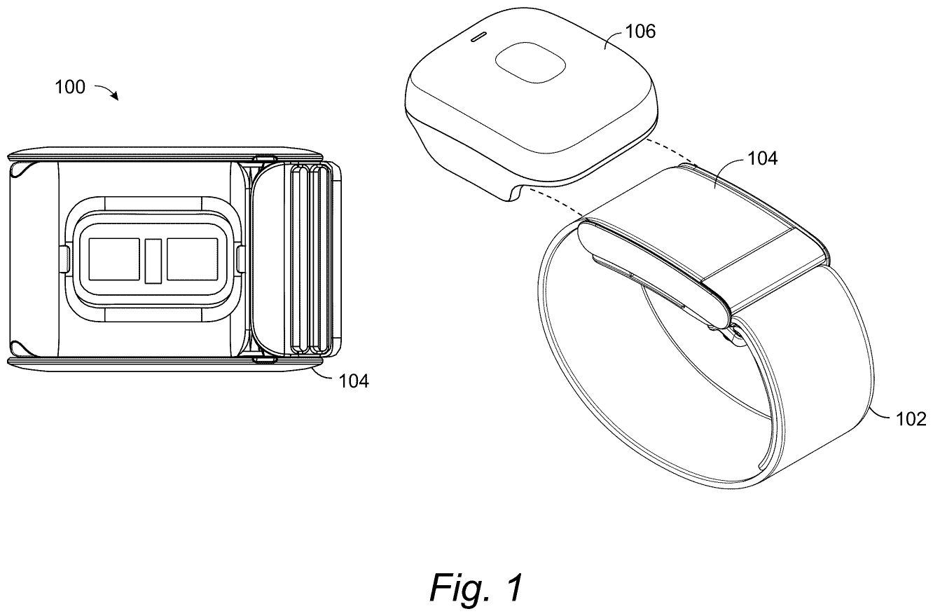

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiogra any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

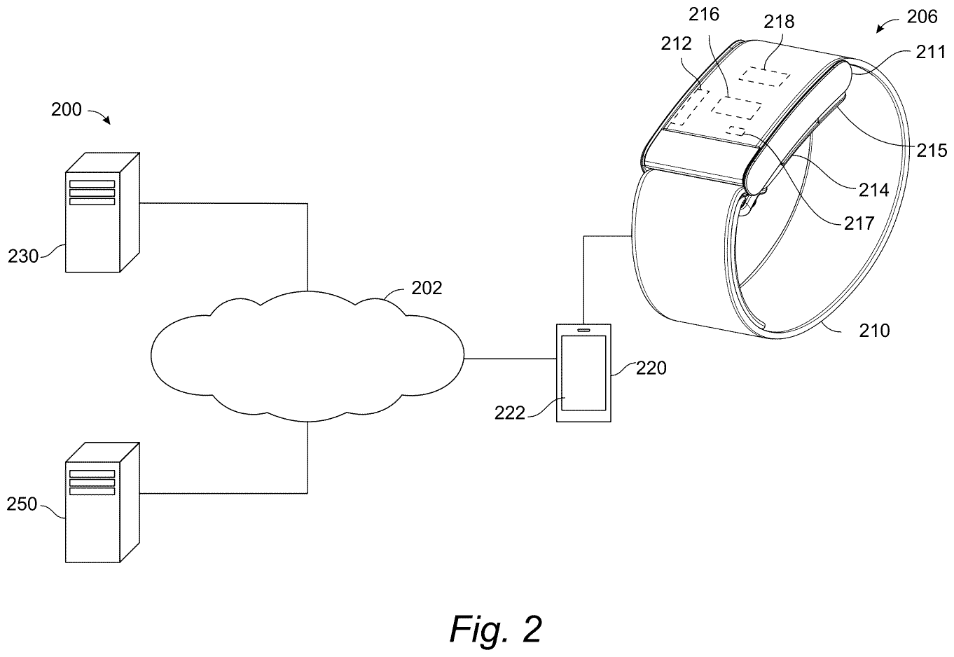

FIG. 2 illustrates a physiological monitoring system. More specifically, FIG. 2 illustrates a system 200 for physiological monitoring that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220.

Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicated detected motion, and/or beacons or the like to self-identify type, weight, and so forth, in order to facilitate automated detection and tracking of exercise activity with other connected devices.

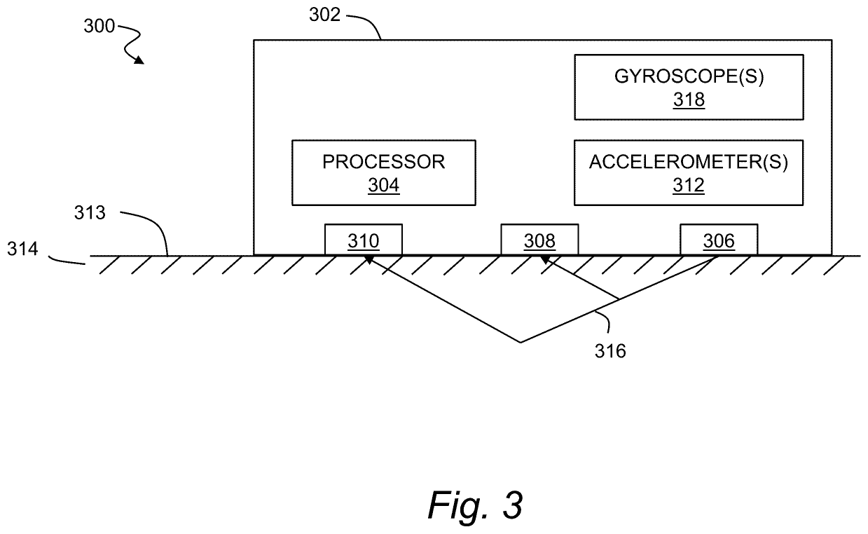

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like such as audio earbuds, earrings or similar, glasses and/or other eyewear, a ring, a headband, and so forth.

The processor 304 may be any microprocessor, microcontroller, application specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware-such as one or more gyroscopes 318, inertial motion sensors, and/or other micro-electromechanical system (MEMS) sensors-may also or instead be used for these purposes. More generally, the physiological monitor 302 may include any additional components, subsystems, and the like suitable for supporting various modes of physiological monitoring and contextual data acquisition as described herein.

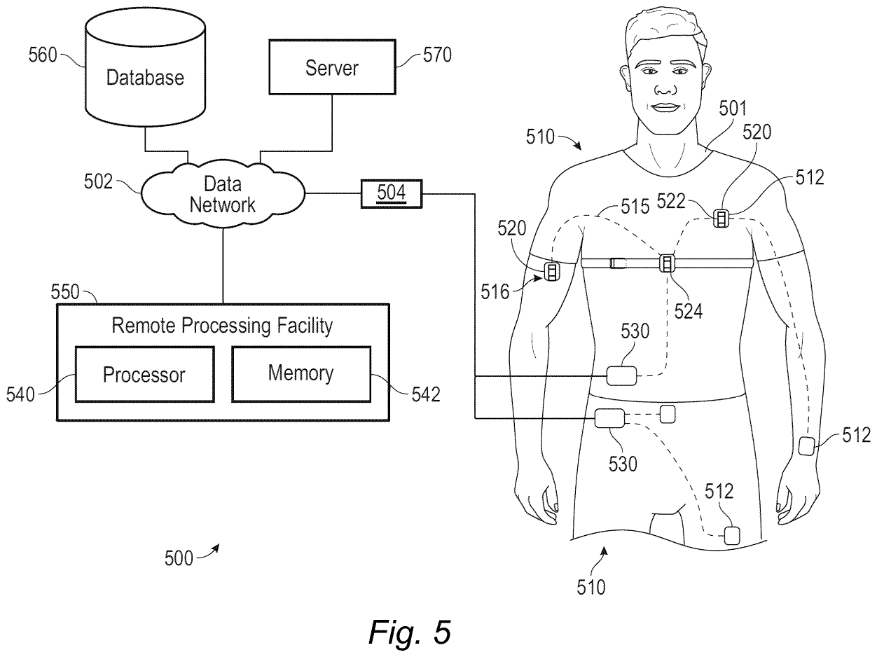

The physiological monitors described herein-e.g., in the systems described above or elsewhere herein-may be provided in one or more different form factors. That is, although a wrist-worn device is illustrated in FIGS. 1 and 2, and garments with sensors are illustrated in FIG. 5, other form factors are also or instead possible, some of which are discussed below by way of example.

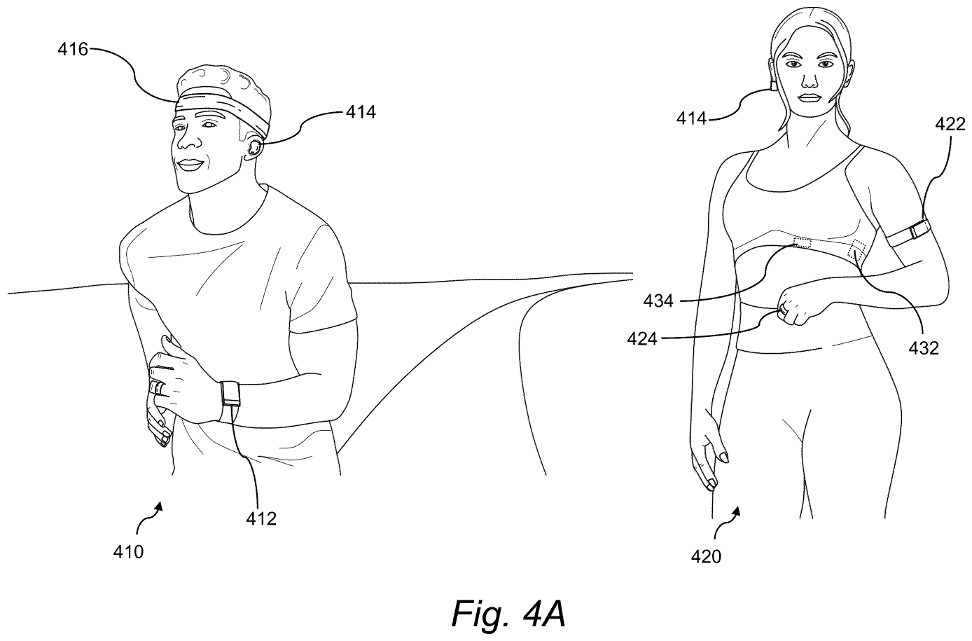

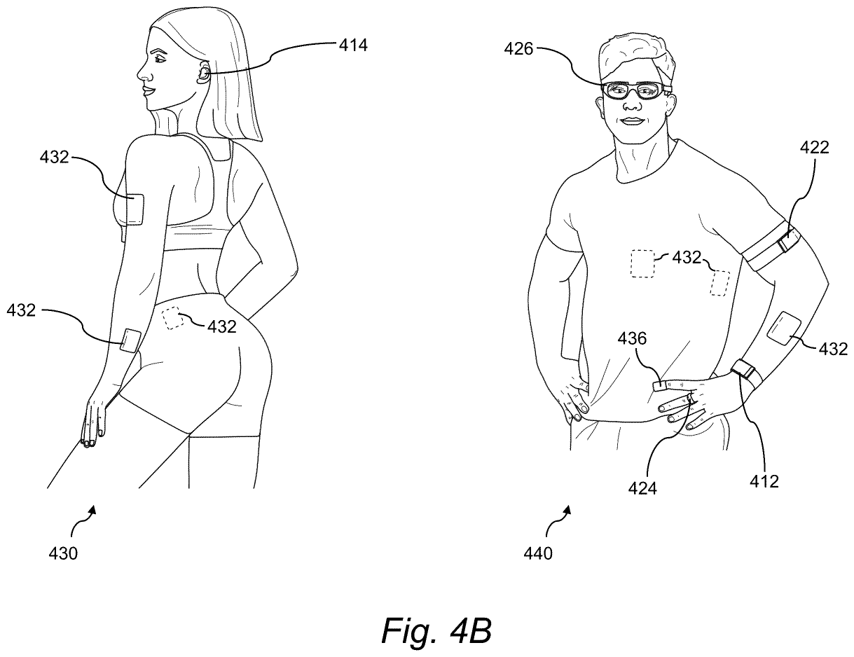

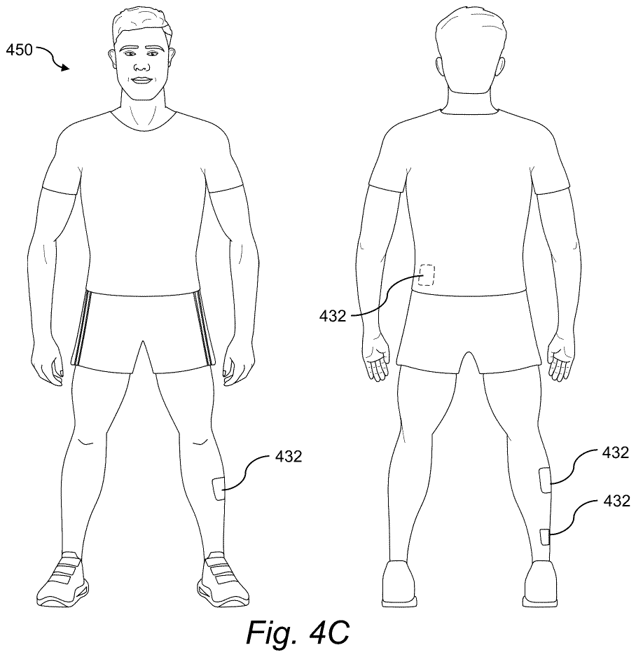

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth.

These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a finger-tip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The finger-tip monitor 436 may include a clamp, clip, or the like, and may be the same or similar to any of the physiological monitors described herein. In some aspects, the finger-tip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiogramar blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-ﬁ or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these.

The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiogramalectroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

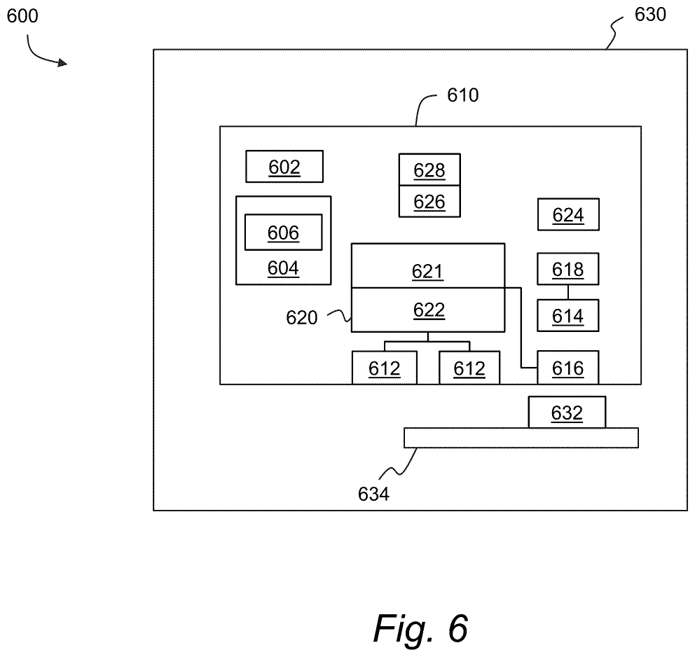

FIG. 6 shows a system for transitioning between power modes for an electronic device using packaging thereof. Batteries of electronic devices may become depleted during shipping and/or storage, i.e., before the first use thereof by an end user. This situation can arise when devices consume power while in transit and can result in user dissatisfaction when devices are not ready for immediate use upon arrival. As a significant advantage, the techniques described herein can mitigate in-transit battery depletion, thus ensuring quick and easy activation for end-users upon receipt, while also preventing unintended reentry into low power mode during normal use. These techniques may be used, e.g., with any of the physiological monitoring devices described herein, or any other electronics device(s) that might usefully be shipped in a low power (but not off) mode.

It will be understood that the term “low power mode” as used herein shall generally refer to an operational state of a device in which power consumption is reduced relative to a normal operating mode. This may include, for example, reducing clock speeds, disabling certain components or sensors, and/or limiting functionality to conserve battery life.

The low power mode may be used during shipping and/or storage to preserve battery charge during a period of prolonged non-use, e.g., so that the battery retains a substantial useable charge upon first use by an end user. This is distinguished from a high power mode or operating mode, during which the device is used as intended. While a device may also or instead have various battery-saving, low power modes, the “low power mode” contemplated herein has a number of distinguishing characteristics including long term battery preservation during non-use, disablement and/or absence of ordinary operating functions or features, and an irreversible transition to a normal power mode that can be physically initiated by an end user. By way of example, a low power mode for a device may disable various sensors, communications, and input/output hardware, and may use a reduced clock speed relative to the normal operating mode, and may be terminated when an end user removes the device from packaging, e.g., using any of the techniques described herein. The low power mode may be interchangeably referred to herein as the shipping mode or factory mode.

The “normal power mode” generally refers to the primary operational state of a device in which functionality of the device is enabled and the device consumes more than the minimal power of the low power mode. Transitioning to the normal power mode may, for example, include activating sensors or other hardware, increasing a processor clock speed, enabling communications and user input/output, and so forth. The normal power mode may thus be any operating mode for active use of an electronic device by an end user, and may include various consumer-controlled or device-controlled power consumption tiers such as a full power mode, a battery-saver mode, an airplane mode, and so forth.

It will be understood that “permanently disabling” a device or “irreversibly terminating” the lower power mode, or any similar phrases used herein, is intended to refer to a transitioning out of the low power mode in a manner that cannot be reversed without mechanical intervention or a factory reset or the like. In the context of the present disclosure, this may involve deleting code for low power mode operation, physically disconnecting low power mode circuitry, and/or otherwise ensuring that the device cannot reenter the low power mode after initial activation.

The system 600 generally includes a device 610 such as any of the wearable physiological monitors or other devices described herein, or any other consumer electronics that might usefully be packaged in a low power mode, as well as packaging 630 such as any consumer packaging structurally configured to receive the device 610, e.g., for shipping, storage, shelf display, and the like. The device 610 may include one or more sensors 612 for monitoring or other use of the device 610, a waking module 614, one or more sensors 616 for detecting unpackaging of the device 610 (e.g., a Hall effect sensor), a transition module 618, a sensing circuit 620, a low power mode circuit 624, a battery 626, and a wireless charging interface 628. The packaging 630 may generally include a sensor trigger 632 (e.g., a disposable magnet) and a removable insert 634, as well as any packaging or packaging materials suitable for securing the device 610 in the packaging 630 during storage, shipping, and so forth.

The one or more sensors 612 for monitoring or other use of the device may include any of the sensors or other sensing circuitry described herein, e.g., for physiological monitoring, environmental monitoring, or any other sensing functions. For example, the one or more sensors 612 may include one or more sensors for physiologically monitoring a wearer of the device 610. To this end, the one or more sensors 612 may include one or more light emitters for emitting light at one or more desired frequencies toward a user's skin, and one or more light detectors for received light reflected from the user's skin (e.g., where such light detectors may include a photo-resistor, a phototransistor, a photodiode, and the like). More generally, the one or more sensors 612 include any sensors, sensing circuitry, input/output hardware, and the like that might consumer power during normal operation of the device 610.

The device 610 may be configured to operate in a low power mode and a normal power mode, and to transition from the low power mode to the normal power mode under various conditions. In the normal power mode, the device 610 may drive the one or more sensors 612 or otherwise power the device 610 for normal use. In the low power mode, the device 610 may operate with reduced functionality and reduced power consumption relative to the normal power mode. By way of example, the low power mode may use a reduced clock speed compared to the normal power mode, e.g., in order to decrease the rate at which a processor 602 of the device 610 executes instructions, thereby reducing the energy usage of the device 610. The device 610 may also disable or reduce various operating functions such as sensing, processing user input/output, communications, and so forth. The device 610 may be configured to transition from the low power mode to the normal power mode in response to a signal, such as a signal from the waking module 614 described herein.

The waking module 614 may include, or otherwise be in communication with, the one or more sensors 616 for detecting unpackaging of the device 610. The waking module 614 may be configured to detect a condition—e.g., a waking condition—for transitioning from the low power mode to the normal power mode using data or signals from the one or more sensors 616. In response to detecting the waking condition, the waking module 614 may provide a control signal to the device 610 for the device 610 to initiate a transition from the low power mode to the normal power mode.

The waking module 614 may, for example, include a microcontroller that can receive and interpret a signal from a Hall effect sensor, capacitive sensor, infrared sensor, or other contact or proximity sensor or the like used as the one or more sensors 616 for detecting unpackaging of the device 610. The waking module 614 may usefully incorporate operational amplifiers, comparators, and other active or passive signal conditioning electronics to condition sensor signals from the one or more sensors 616 before interpreting the sensor signal(s) and generating a responsive control signal to transition the device 610 to the normal power mode.

The waking module 614 may also or instead use firmware or other executable instructions to filter and process the sensor signal(s), and to analyze the sensor signal(s) for the presence of a waking condition for the device 610 to transition from the low power mode to the normal power mode. The firmware may also contain logic for handling multiple types of sensors and prioritizing their signals to ensure accurate detection of the triggering event. In general, the waking module 614 may advantageously be deployed in a minimal hardware configuration that provides electrical power for solely detecting a trigger from the one or more sensors 616. This can help to reduce power consumption and avoid depleting the battery 626 on the device 610 while monitoring over extended periods of time.

In one aspect, the one or more sensors 616 may include a Hall effect sensor, and the waking module 614 may be configured to detect a condition for transitioning from the low power mode to the normal power mode based on the movement of a fixed magnet near the Hall effect sensor. The resulting electrical signal from the Hall effect sensor may be detected by the waking module 614 and interpreted as a waking condition, in response to which the waking module 614 may provide a corresponding control signal to the device 610 for the device 610 to transition from the low power mode to the normal power mode.

The one or more sensors 616 for detecting unpackaging may also or instead include other sensors. For example, the one or more sensors 616 may include one or more optical sensors, e.g., where a light sensor in the device 610 detects when it is removed from light-blocking aspects of the packaging 630; one or more motion sensors, e.g., to detect movement or orientation changes when the device 610 is removed from the packaging 630; one or more pressure sensors, e.g., where a change in pressure may be detected when the device 610 is removed from packaging 630 that is pressurized or vacuum sealed; one or more capacitive sensors, e.g., to detect changes in the surrounding electromagnetic field when the device 610 is touched or removed from the packaging 630; an RFID (radio frequency identification) or NFC (near field communication) sensor, e.g., an RFID tag communicatively coupled to an RFID tag reader in the packaging 630 that triggers a transition when the device 610 moves out of range; a mechanical switch, e.g., a mechanical switch that is depressed or otherwise retained in a closed or open position while the device 610 is in the packaging 630 and released upon removal of the device 610; a piezoelectric sensor; an audio sensor; a proximity sensor; a barometric sensor; a photodiode; a strain gauge; and so forth.

One or more of these configurations may be used to advantageously detect an initial user intent to begin using the device 610 without requiring specific set up or power on steps by the user. However, it will also be appreciated that, while the one or more sensors 616 are described as detecting an unpackaging event, the one or more sensors 616 may also or instead be configured to detect any event suitable for triggering a transition from a low power mode to a normal power mode, such as a user pressing a button for the first time or the device 610 being placed in a charging cradle for the first time. Furthermore, placing hardware such as a stand-alone, fixed magnet in the packaging 630 for use as a trigger advantageously simplifies the overall sensing architecture by removing the requirement for wires and circuitry within the disposable packaging. However, it will be appreciated that the specific arrangements described herein, such as a Hall effect sensor on the device 610 and a magnet in the packaging 630, can also be deployed in a complementary configuration. For example, the device 610 may include a magnet, and the packaging 630 may include a Hall sensor that detects movement of the magnet on the device 610 and generates a control signal that is transmitted to the device 610. This may be useful, e.g., in packaging where the device 610 is tethered to the packaging 630 with a connection that can provide a wire for the control signal. More generally, while various embodiments are described herein, numerous alternative techniques may be used consistent with the general approach described herein for deploying an unpackaging wake up system. All such alternative embodiments and configurations are intended to fall within the scope of this disclosure.

In another aspect, the one or more sensors 616 for detecting unpackaging of the device 610 may also be used during normal operation of the device 610, e.g., for physiological sensing in the normal power mode. For example, where the one or more sensors 616 for detecting unpackaging including one or more optical sensors, these same sensors may also be used to acquire photoplethysmography or pulse oxygenation data during use of the device 610. Thus, the one or more sensors 612 for monitoring may include or may be the same as the one or more sensors 616 for detecting unpackaging of the device 610 in some embodiments.

The transition module 618 on the device 610 may be configured to respond to the control signal from the waking module 614 by causing the device 610 to transition to a normal power mode for its intended use. This transition may include powering up of the device 610 for use, as well as permanently disabling the low power mode on the device 610 in order to prevent an inadvertent return to the dormant or low power state. Thus, the transition module 618 may more generally manage the process of switching from a low power shipping mode to a normal operating mode. To this end, the transition module 618 may coordinate the actions of the processor 602, the memory 604, the waking module 614, and/or sensors of the device 610 to ensure a smooth and permanent transition. Once the transition is complete, the transition module 618 may prevent a return to the low power mode, e.g., by permanently disabling a low power mode circuit 624. In general, the low power mode circuit 624 may include any circuitry supporting low power operation of the device 610 in the low power mode. While illustrated as separate circuitry, it will be appreciated that the low power mode circuit 624 may be, or may include, the waking module 614, the one or more sensors 616 for detecting unpackaging, and so forth. Permanently disabling the low power mode may also or instead include deleting code 606 stored in the memory 604 of the device 610 that configures the processor 602 of the device 610 to operate in the low power mode. Alternatively, permanently disabling the low power mode may include irreversibly and/or mechanically decoupling circuitry for the low power mode from the device 610, e.g., by fusing a switch closed or open within the low power mode circuit 624, storing kill codes to disable low power functions, storing bypass code or instructions in a one-time programmable memory, and so forth.

The transition module 618 may include one or more of: a microcontroller designed to manage power states, a dedicated power management integrated circuit (PMIC) that controls the switching between the low power mode and the normal power mode, non-volatile memory components that store the state of the power mode, hardware interlocks such as permanent mechanical fuses or relays may be used to ensure the low power mode cannot be re-entered once disabled, firmware programmed into a microcontroller that executes the state transition logic (e.g., code 606 for detecting signals from the waking module 614 and executing the transition to a normal power mode), code for permanently disabling the low power mode (such as code that deletes or overwrites portions of the firmware responsible for low power mode operation), software interlocks that prevent re-entry into the low power mode by setting flags or modifying configuration registers may also be part of software components of the transition module 618, and the like.

As described herein, the sensing circuit 620 for the device in the operating or normal power mode may also include some or all of the waking module 614 circuitry and or the transition module 618 circuitry. More generally, the sensing circuit 620 may, include both (i) circuitry and/or other components for sensing a condition to transition the device 610 from the low power mode to the normal power mode, and (ii) circuitry and/or other components for other sensing, such as sensing that occurs while the device 610 is in the normal power mode (e.g., sensing for physiological monitoring as described above). In other aspects, the sensing circuit 620 may be separate and distinct from a low power mode circuit 624 that includes circuitry and/or other components for operating the device 610 in the low power mode and/or sensing a condition to transition the device 610 from the low power mode to the normal power mode, where the sensing circuit 620 includes circuitry and/or other components for sensing that occurs while the device 610 is in the normal power mode. Additionally, the sensing circuit 620, waking module 614, transition module 618, and so forth may be separate integrated circuits, or may be integrated into a single device or combination of devices, particularly where the device(s) support low power and/or low clock rate modes that can be used to conserve power while in the low power mode.

As noted above, the one or more sensors 616 for detecting unpackaging may be the same as the one or more sensors 612 for monitoring or different than the one or more sensors 612 for monitoring. Where the one or more sensors 616 for detecting unpackaging are different, the one or more sensors 616 for detecting unpackaging and the one or more sensors 612 for monitoring may be coupled to the device 610 (and/or the sensing circuit 620) in two parallel circuits including a first parallel circuit 621 for the low power mode and a second parallel circuit 622 for the normal power mode. When the device 610 is in the low power mode, the sensing circuit 620 may disconnect the second parallel circuit 622 for the normal power mode and solely operate with the first parallel circuit 621 for the low power mode. In this manner, circuitry for driving and monitoring the sensors may advantageously be reused for both low power and normal power modes. Alternatively, where a completely separate low power mode circuit 624 is used, the device 610 may operate in a low power mode by disconnecting power to the sensing circuit 620 of the device 610, so that power is only used by the low power mode circuit 624.

The battery 626—and/or other internal components of the device 610—may be disposed in a sealed enclosure, e.g., a waterproof enclosure and/or a hermetically sealed enclosure for the device 610. The device 610 may further include a wireless charging interface 628 (such as a wireless power interface) for the battery 626 that permits the battery 626, and thus the device 610, to be charged without physical connectors. The battery 626 may provide the power needed for the device 610 to operate in one or both of the low power and normal power mode, or the device 610 may include separate batteries or power source hardware for the low power and normal power mode. This permits various battery configurations, such as a sacrificial or disposable small, low self-discharge rate, low amp hour battery or super capacitor for the low power mode, coupled with a higher energy storage battery for regular use in the normal power mode.

The packaging 630 may generally be shaped, sized, and structurally configured to receive and contain the device 610 (and optionally one or more accessories thereof) for storage, shipping, or shelf display. The packaging 630 may include a sensor trigger 632 configured to cooperate with the one or more sensors 616 of the device 610 in order to support detection of unpackaging of the device 610. By way of example, the sensor trigger 632 may include a magnet (e.g., a disposable magnet) positioned in the packaging 630 to stimulate the one or more sensors 616 (e.g., which may be a Hall effect sensor) to generate an electrical signal when the device 610 is moved relative to the packaging 630, thus detecting a condition for transitioning from the low power mode to the normal power mode.

Other configurations are also possible for the sensor trigger 632 and the one or more sensors 616 for detecting unpackaging. For example, the sensor trigger 632 may be a component that cooperates with any of the one or more sensors 616 described herein to create a signal when the device 610 is moved relative to the packaging 630. In another aspect, the one or more sensors 616 for detecting unpackaging may operate without a hardware trigger. For example, where the one or more sensors 616 includes a light sensor to detect when the device 610 is removed from light-blocking aspects of the packaging 630, the unpackaging condition may be implicitly detected based on the presence of light. In this case, the packaging 630 may provide a sensor trigger 632, e.g., with a portion of the packaging 630 that shields the one or more sensors 616 from ambient light. While a variety of such configurations are possible, it will also be appreciated that a detection system with a Hall effect sensor and a fixed magnet advantageously provides a highly reliable, low power, inexpensive detection mechanism suitable for use with disposable packaging of consumer products.

The device 610 may include a processor 602 and a memory 604, which may implement one or more of the sensing circuit 620, the low power mode circuit 624, and so forth, or may be configured separately therefrom to support normal operations of the device 610 such as physiological monitoring or other user functions. More generally, the memory 604 may be any memory for non-transitory storage of computer code, data, and the like, and may include code 606 that is executable by the processor 602 to perform one or more operations in the system 600. By way of example, the code 606 may include instructions to perform one or more monitoring and/or sensing operations of the device 610 as described herein (e.g., with respect to one or more of FIGS. 1-5 above). The code 606 may also or instead include instructions to perform one or more of the following: a transition between different power modes, disabling one or more power modes, running one or more power modes, and the like.

In one aspect, the device 610 may be configured, e.g., via the transition module 618, to respond to a sensor signal from the one or more sensors 616, and/or a control signal from the waking module 614, to transition from the low power mode to the normal power mode. In one aspect, this may include permanently disabling the low power mode on the device 610. For example, this may include deleting the code 606 stored in the memory 604 of the device 610 that configures the processor 602 to execute in the low power mode. Permanently disabling the low power mode may also or instead include mechanically decoupling circuitry for the low power mode from the device 610—e.g., the low power mode circuit 624—or otherwise irreversibly disabling, decoupling, or removing low power mode capabilities from the device 610.

The sensor trigger 632 of the packaging 630 may trigger the transition from a low power mode to a normal power mode. For example, when the packaging 630 is opened and/or certain pieces of the packaging 630 are displaced in a predetermined manner, the sensor trigger 632 may interact with the one or more sensors 616 to generate a signal to the waking module 614 indicating that the device 610 should transition to the normal power mode. In one example, the device 610 is placed within the packaging 630 such that a Hall effect sensor of the device 610 is within a magnetic field of a disposable magnet in the packaging 630, or within a path of the magnetic field during unpackaging, such that the magnet moves relative to the Hall effect sensor (or vice-versa), thereby stimulating the Hall effect sensor to generate a responsive electrical signal to the waking module 614. The waking module 614 can respond to this signal by generating a control signal to the transition module 618 causing the transition module 618 to initiate a transition of the device 610 from the low power mode to the normal power mode.

In one aspect, the packaging 630 may include a removable insert 634 that is coupled to or otherwise incorporates the sensor trigger 632 (e.g., a magnet or the like). This removable insert 634 may be designed to fit securely within the packaging 630, ensuring that it remains in place during transit. The removable insert 634 may be manually removed by a user when unpackaging the device 610, thus causing displacement of the sensor trigger 632 relating to the one or more sensors 616 in a manner that causes the one or more sensors 616 to generate a responsive signal. For example, the removable insert 634 may be positioned within the packaging 630 so that a user must manually move the removable insert 634 in order to access the device 610 or remove the device 610 from the packaging 630. In another aspect, the device 610 may be removed from the packaging 630 with the removable insert 634, and then a user may subsequently remove the removable insert 634 from the device, thus causing displacement of the sensor trigger 632 relative to the one or more sensors 616 to generate the responsive signal. The removable insert 634 may include a pull-tab or the like, which may be labeled or otherwise visually marked in order to direct the user to remove the removable insert 634 from the device 610.

The packaging 630 can be constructed from one or more various types of materials such as paper, cardboard, wood, plastic, rubber, metal, biodegradable materials, and combinations thereof, and so on, as well as combinations of any of the foregoing. The packaging 630 may be configured into a plurality of shapes and sizes—e.g., the packaging 630 can range from a simple rectangular box to more complex, custom-shaped designs that snugly fit the contours of the device 610 and/or its accessories, providing additional protection during transit. For instance, the packaging 630 may include a rectangular box having internal compartments and/or inserts to hold the device 610 securely in a predetermined configuration and to prevent undesired movement thereof.

Different opening mechanisms for the packaging 630 can be used depending on the device and the intended user experience, and may include doors, removable covers, hinged covers, inserts, liners, wrappers, and so forth. In one aspect, the opening mechanism can be structurally configured for use with a device 610 having a one or more sensors 616 configured for predetermined cooperation with a sensor trigger 632 in the packaging 630 to facilitate a transition from a low power mode to a normal power mode during unpackaging. Such opening mechanisms may include: flap-top boxes, slide-out boxes, tear strips, clamshell packaging, magnetic closures, and the like. Each of these packaging designs can be customized to include the components for the disclosed system 600, such as a disposable magnet for activating a Hall effect sensor when the packaging 630 is opened or when the device 610 is removed from the packaging 630. By configuring the packaging 630 and device 610 in this manner, the system 600 can transition from a low power mode to a normal power mode as the device 610 is unpackaged, thus helping to ensuring that the device 610 is ready for use when it is received by the end user.

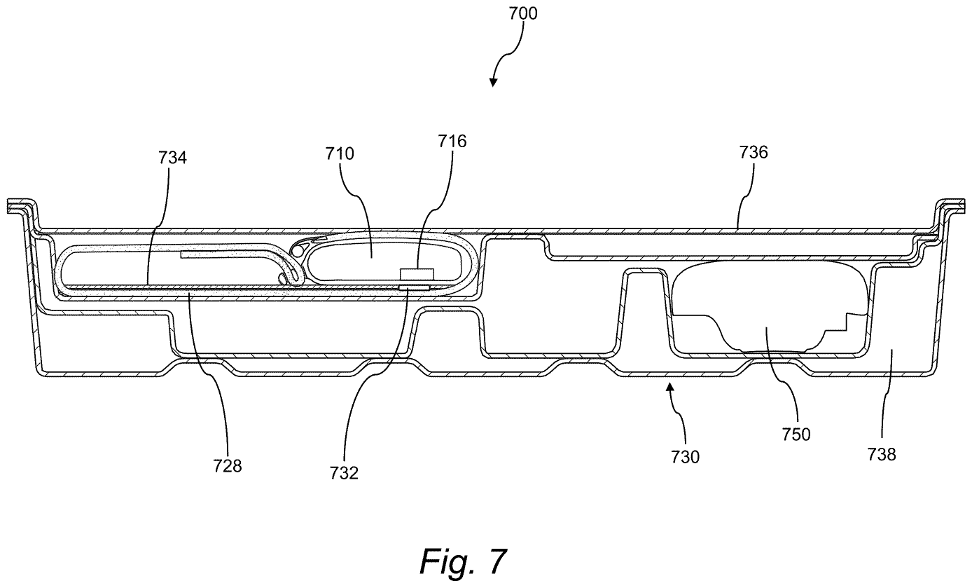

FIG. 7 is a cross-sectional view of an electronic device in its packaging. In general, the system 700 may include a device 710 disposed in packaging 730 for shipping or storage. The device 710 may be any of the electronic devices described herein, such as any of the physiological monitors described with reference to FIGS. 1-6, or any other device that ships with a battery and is preferably ready for user deployment when received.

In FIG. 7, the packaging 730 is shown as having cross-hatching, where the packaging 730 is generally structurally configured to protect the device 710 and any accessories (e.g., an external battery 750) from damage during shipping and/or storage, and may include specific compartments for different components, e.g., to mitigate movement and minimize the risk of damage during transit. These compartments can be created using, for example, molded pulp trays or dividers, custom foam inserts, and the like, where such components may be sized and shaped to fit the shape and contours of other contents of the packaging 730. This approach can ensure a snug fit and provide relatively sturdy support for electronic items. Additionally, the packaging 730 may incorporate multiple layers, such as a top container for the device 710 and a bottom container for the external battery 750, where each is enclosed within a larger outer container component. This multi-layered approach can offer protection against impacts, vibrations, and other potential hazards that electronic products might encounter during shipping and handling. Further, to retain the device 710 and other objects, the packaging 730 may include packing material in interstitial spaces 738 that wholly or partially fills one or more these interstitial spaces 738 or compartments of the packaging 730—e.g., to maintain relative positions of the contents of the packaging 730.

Thus, in general, the packaging 730 may be designed to house and protect the device 710 during shipping and storage. The packaging 730 may further contain accessories for the device 710 and so forth. By way of example, the packaging 730 may contain a strap 728 for the device 710 (where the strap 728 is shown in the figure having stipple shading for clarity), an external battery 750 for wireless recharging of the device 710, user information cards, and so forth.

The device 710 may be positioned within a compartment of the packaging 730 in a predetermined manner. For example, as shown in the figure, the device 710 may be disposed adjacent to a removable insert 734, which is shown having tightly-spaced cross-hatching in the figure (i.e., as opposed to the cross-hatching for the other portions of the packaging 730). The removable insert 734 may include a sensor trigger 732 (e.g., a disposable magnet) such that a sensor 716 of the device 710 communicates, engages, or otherwise cooperates with the sensor trigger 732. In this manner, when a lid 736 of the packaging 730 is removed and the device 710 is removed from the packaging 730, the displacement of the device 710 relative to the sensor trigger 732 can cause the sensor 716 of the device 710 to detect the unpackaging and generate a trigger for the device 710 to transition from a low power mode to a normal power mode.

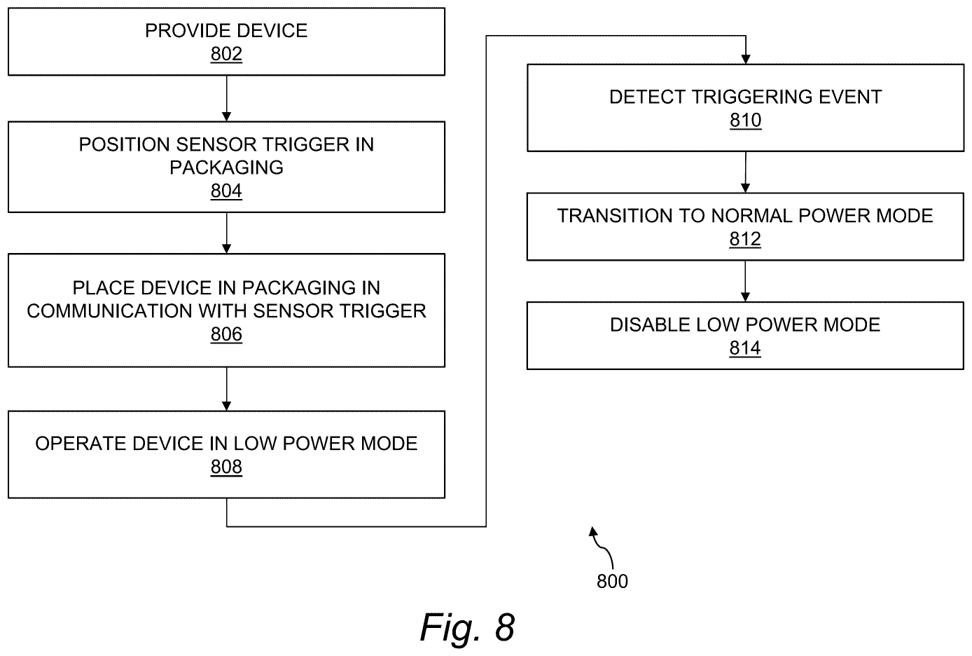

FIG. 8 is a flow chart of a method for managing power modes of an electronic device. The method 800 may generally include transitioning a device from a low power mode (e.g., for shipping or storage) to a normal power mode (e.g., for normal use by an end user) upon removal from packaging. This transition may include, or may be followed by, a permanent disabling of the low power mode. While the method 800 described herein emphasizes the use of packaging-related triggers for transitioning between power modes, the method 800 may also or instead be deployed with other triggering mechanisms, such as ambient light detection, predetermined user interactions with the device, detection of physiological signals, and so on. The method 800 may be performed using any of the systems and techniques described herein.

As shown in step 802, the method 800 may include providing a device, such as a wearable physiological monitor or any of the other devices described herein. In one aspect, the device may include a processor and a memory, and/or any other circuitry suitable for performing the steps described herein. The processor may be configured by computer executable code stored in the memory to operate the device in a low power mode and a normal power mode. Further, the processor may be configured to transition from the low power mode to the normal power mode in response to a signal from a sensor, such as a Hall effect sensor. As noted above, one or more other circuits may also or instead be provided in order to monitor the Hall effect sensor or other sensor(s) for triggering events such as unpackaging of the device or the like.

As shown in step 804, the method 800 may include positioning a sensor trigger in packaging for the device. By way of example, the sensor trigger may include a magnet positioned in the packaging to stimulate a Hall effect sensor in the device when the device is removed from the packaging. More specifically, the sensor trigger may include a disposable magnet affixed to a removable insert within the packaging. However, other types of sensor triggers (and/or cooperating sensors on the device) are also or instead possible, and other placements within the packaging are also or instead possible. For example, the sensor trigger may be integrated into the packaging material itself, or may be a separate component placed within a compartment of the packaging. In some aspects, the packaging completely lacks a sensor trigger, and the sensor trigger may be an ambient condition such as light, pressure, touch, and so forth.

As shown in step 806, the method 800 may include placing the device in the packaging, e.g., with a sensor (such as a Hall effect sensor) positioned to interact with the sensor trigger of the packaging (such as a magnet). That is, in an aspect, the packaging may include a magnet positioned to generate the signal with a Hall effect sensor of the device when the device is removed from the packaging. In this manner, the device may be positioned within the packaging such that a Hall effect sensor in the device is in proximity to a magnet in the packaging-e.g., the device may be placed in a specific compartment of the packaging designed to align the device's Hall effect sensor with the magnet. In an aspect, a removable insert having the sensor trigger (e.g., magnet in this example), is placed in engagement with the device, and then the device is inserted into the packaging. However, it will be understood that other forms of placing the device, and a sensor thereof, in communication with the sensor trigger of the packaging are also or instead possible. For example, the device may be placed in packaging in a manner that allows for wireless communication with the sensor trigger, or the device may be connected to the sensor trigger through a temporary physical connection.

It will be appreciated that the sensing trigger may be configured in various ways so that it only detects a triggering event after the device is placed in the packaging. For example, a sensing circuit may be configured so that a first sensed event (e.g., movement of a magnet relative to a Hall effect sensor) arms the sensing circuit as the device is placed into the packaging. In another aspect, the device or the sensing circuit may be activated remotely once the device is placed into the packaging or otherwise positioned adjacent to a sensor trigger such as a magnet. A variety of circuits and techniques are known in the art for performing such arming and triggering functions. In general, any such technique(s) suitable for arming the device to detect unpackaging after the device has been positioned in the packaging may be used to facilitate placement of the device in the packaging as described herein.

As shown in step 808, the method 800 may include operating the device in a low power mode. This may, for example, include placing the device in the packaging while the device is in the low power mode, or transitioning the device to the low power mode after the device has been placed into the packaging. As described herein, the device may be configured to operate in a low power mode, e.g., with reduced functionality and power consumption, until a triggering event. In the low power mode, certain sensors, processors, and other circuitry and the like may be disabled, particularly any circuitry not necessary or helpful for detecting the triggering event or condition for the device to transition to the normal power mode. Other techniques may also or instead be used in the low power mode to conserve power and extend battery life. For example, the low power mode may employ a reduced clock speed for a processor or other clocked circuitry executing on the device. The lower power mode may also check for a waking event on a reduced schedule, such as once every few seconds. In this case, the detection circuitry for detecting the triggering event may also usefully record the triggering event in a non-volatile or non-transitory manner so that evidence of the triggering event is available when the processor or other circuitry polls or otherwise checks for same.

As shown in step 810, the method 800 may include detecting a triggering event. For example, this may include detecting the motion of a fixed magnet in packaging for the device relative to a Hall effect sensor in the device, or otherwise detecting opening of the packaging for the device, movement of the device relative to the packaging or any other associated user manipulation of the packaging and/or the device. For example, this may include detecting a switch or circuit that opens or closes, detecting proximity or distance with an RFID tag or other wireless tags, sensors, or the like, optical detection of exposure to ambient light, and so forth. More generally, a variety of techniques are known in the art for detecting unpackaging of a device or displacement of the device relative to the packaging, and may be used for detecting a triggering event as contemplated herein. In general, the triggering system may advantageously use low cost, low power circuitry and components such as a small, fixed magnet and a Hall effect sensor.

As shown in step 812, the method 800 may include transitioning the device to a normal power mode. For example, upon detecting a triggering event, the device may activate various functions and enter a normal operating state for an end user. For example, where the device is a physiological monitor, transitioning to a normal power mode may include activating sensors, processors, and other circuitry for monitoring and user interaction. The transition may also or instead include other intermediate steps or modes, e.g., where the device performs initial diagnostics, requests user confirmation of activation, detects an active monitoring state, and so forth.

As shown in step 814, the method 800 may include disabling the low power mode. For example, the device may be programmatically or physically configured to prevent reentry into the low power mode after transitioning to the normal power mode in order to prevent the device from accidentally reverting to the low power mode after activation. Disabling the low power mode may include deactivating the circuitry and/or software processes associated with the low power mode, setting a flag in device memory to prevent future activation of the low power mode, physically disconnecting circuitry and components for the low power mode, and so forth.

In one aspect, disabling the low power mode may include removing the low power mode in a manner that irreversibly disables the low power mode. For example, the device may be configured, responsive to entering the normal power mode, to permanently disable the low power mode by deleting executable code for the low power mode from the memory of the device, disabling or disconnecting circuitry for the low power mode, and so forth. More generally, any of the techniques described herein for disabling functions or components of the low power mode may be used to irreversibly deactivate the low power mode as contemplated herein. In this context, it will be understood that irreversibly deactivating the low power mode more generally refers to any technique rendering it impossible or impractical for an end user to return the device to the low power mode without rebuilding or reprogramming the device. Thus, deleting relevant program code, or disconnecting memory containing low power mode code, or disconnecting or disabling sensing circuitry and other components associated with the low power mode, may serve to irreversibly deactivate the low power mode as contemplated herein.

In one aspect, disabling the low power mode may include disabling the low power mode in a manner such that an end user would not be able to reuse, reapply, or reenter code configured to place the device in the low power mode, although it still may be theoretically possible to reenter this code via a manufacturing command, e.g., at a manufacturing facility or the like. By way of example, the process for placing a device in a low power mode may include: sending the device a command to enter a low power mode state, e.g., for shipping the device; receiving the command at the device, where in response, the device sets a signal high that enables the sensing and triggering circuitry for initiating, maintaining, and/or exiting the low power mode (e.g., Hall effect circuitry or the like, which may be referred to in this context as the low power mode circuitry); and placing the device in packaging near a sensor trigger (e.g., a magnet), where the sensor trigger turns on the low power mode (e.g., a magnet turns on a Hall effect switch) and powers down any normal power mode circuitry. Continuing with this example, when the device is removed from the packaging, the low power mode circuitry switches off (e.g., the Hall effect switches off), and the normal power mode circuitry is powered up, although the device may still remain in low power mode at this point; subsequently, when an end user pairs the device with their phone (or otherwise activates the device), the device exits the low power mode and sets a signal low that disables the low power mode circuitry. Thus, in this example, there is a firmware state for low power that can only be entered via a command at the factory or the like, where, when a device is in that state it will power down in the presence of a sensor trigger (e.g., magnet) and power back up with the removal of the sensor trigger; and once the device is paired or otherwise activated, the firmware exits the low power mode state and is no longer impacted by the presence of a sensor trigger.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure, and the illustrated embodiments are not intended to limit the scope of the present invention as defined by the following claims.

1. A system comprising:
a device having a low power mode and a normal power mode, wherein:
the normal power mode drives one or more sensors for monitoring,
the low power mode provides reduced power consumption relative to the normal power mode, the low power mode including a sensing circuit that uses at least one of the one or more sensors for monitoring, wherein the at least one of the one or more sensors is coupled to the device in two parallel circuits including a first parallel circuit for the low power mode and a second parallel circuit for the normal power mode, and
the device is configured to transition from the low power mode to the normal power mode in response to a signal;
a waking module on the device,
the waking module including a Hall effect sensor,
the waking module configured to detect a condition for transitioning from the low power mode to the normal power mode with the Hall effect sensor, and
in response to detecting the condition, the waking module configured to provide the signal to the device for transitioning from the low power mode to the normal power mode;
a transition module on the device, the transition module configured to respond to the signal by permanently disabling the low power mode on the device; and
packaging for delivering the device to a consumer, wherein:
the device is placed within the packaging, and
the packaging includes a disposable magnet positioned in the packaging as a sensor trigger to stimulate the Hall effect sensor to detect the condition for transitioning from the low power mode to the normal power mode when the device is removed from the packaging.

2. The system of claim 1, wherein the device includes a wearable physiological monitor.

3. The system of claim 1, wherein the device includes a photoplethysmography monitor.

4. The system of claim 1, wherein the low power mode uses a reduced clock speed compared to the normal power mode.

5. The system of claim 1, wherein the low power mode disconnects power to sensing circuitry of the device.

6. The system of claim 1, wherein the device includes a wireless charging interface.

7. A system comprising:
a device having a low power mode and a normal power mode, wherein:
the normal power mode drives one or more sensors for monitoring,
the low power mode provides reduced power consumption relative to the normal power mode, the low power mode including a sensing circuit that uses at least one of the one or more sensors for monitoring, wherein the at least one of the one or more sensors is coupled to the device in two parallel circuits including a first parallel circuit for the low power mode and a second parallel circuit for the normal power mode, and
the device is configured to transition from the low power mode to the normal power mode in response to a signal;
a waking module configured to detect a condition for transitioning from the low power mode to the normal power mode and, in response to detecting the condition, to provide the signal to the device for transitioning from the low power mode to the normal power mode; and
a transition module configured to respond to the signal by transitioning to the normal power mode and permanently disabling the low power mode on the device.

8. The system of claim 7, wherein the low power mode includes a sensing circuit containing at least one sensor different than the one or more sensors for monitoring.

9-10. (canceled)

11. The system of claim 7, wherein the at least one of the one or more sensors includes a photodetector responsive to a predetermined range of wavelengths.

12. The system of claim 7, wherein the one or more sensors for monitoring include one or more sensors for physiologically monitoring a wearer of the device.

13. The system of claim 7, wherein the low power mode uses a reduced clock speed compared to the normal power mode.

14. The system of claim 7, wherein the low power mode disconnects power to sensing circuitry of the device.

15. The system of claim 7, wherein the device includes a wearable photoplethysmography monitor.

16. The system of claim 7, wherein the device includes a battery sealed in a waterproof enclosure and a wireless charging interface for the battery.

17. The system of claim 7, further comprising packaging for providing the device to a user, wherein the device includes a Hall effect sensor, and wherein the packaging includes a disposable magnet positioned in the packaging as a sensor trigger to satisfy the condition for transitioning by stimulating the Hall effect sensor when the device is removed from the packaging.

18. The system of claim 7, wherein permanently disabling the low power mode includes deleting code stored in a memory of the device that configures a processor of the device to execute in the low power mode.

19. The system of claim 7, wherein permanently disabling the low power mode includes irreversibly and mechanically decoupling circuitry for the low power mode from the device.

20. A method comprising:
providing a device,
the device including a Hall effect sensor, a processor, and a memory, the processor configured by computer executable code stored in the memory to operate the device in a low power mode and a normal power mode,
the normal power mode driving one or more sensors for monitoring,
the low power mode including a sensing circuit that uses at least one of the one or more sensors for monitoring, wherein the at least one of the one or more sensors is coupled to the device in two parallel circuits including a first parallel circuit for the low power mode and a second parallel circuit for the normal power mode, and
the processor configured to transition from the low power mode to the normal power mode in response to a signal from the Hall effect sensor; and
placing the device in packaging, wherein:
the packaging includes a magnet positioned as a sensor trigger to generate the signal with the Hall effect sensor when the device is removed from the packaging,
the device is placed in the packaging in the low power mode, and
the device is configured, responsive to entering the normal power mode, to disable the low power mode by deleting executable code for the low power mode from the memory of the device.

21. The method of claim 20, wherein the at least one of the one or more sensors includes a photodetector responsive to a predetermined range of wavelengths.

22. The method of claim 20, wherein the low power mode uses a reduced clock speed compared to the normal power mode.
